(19) United States
(12) Patent Application Publication
(10) Pub. No.: US 2021/0180001 A1
(43) Pub. Date: Jun. 17, 2021

(54) BIOREACTOR WITH FREEZE-THAW CAPABILITIES TO ENHANCE PRODUCT RECOVERY AND RELATED METHODS

(71) Applicant: UNIVERCELLS TECHNOLOGIES S.A., Nivelles (BE)

(72) Inventors: José Castillo, Brussels (BE); Bastien Mairesse, Uccle (BE); Sebastien Jean-Pierre Michel Rodriguez, Ecaussines-Lalaing (BE)

(21) Appl. No.: 17/048,769

(22) PCT Filed: Apr. 23, 2019
(86) PCT No.: PCT/EP2019/060359

§ 371 Date: Oct. 19, 2020

Related U.S. Application Data
(60) Provisional application No. 62/661,413, filed on Apr. 23, 2018.

(51) Int. Cl. C12M 1/02; C12M 1/12

(52) U.S. Cl. CPC C12M 41/22; C12M 25/18

(57) ABSTRACT

An apparatus is provided for bioprocessing, such as for culturing cells. The apparatus includes a bioreactor with a chamber having cells and a chamber for) temperature regulation of the cells. In some embodiments, a freezer is connected to the bioreactor for freezing the cells in the chamber, and a heater may also be provided for actively thawing the cells. Related methods are also disclosed.

This application claims the benefit of U.S. Provisional Patent Application Ser. No. 62/661,413, the disclosure of which is incorporated herein by reference. The disclosures of U.S. Patent Application Publication No. 2018/0282678, International Patent Application PCT/EP2018/076354, U.S. Provisional Patent Application 62/711,070, and U.S. Provisional Patent Application 62/725,545 are incorporated herein by reference.

### TECHNICAL FIELD

This document relates generally to the cell culturing arts and, more particularly, to a bioreactor provided with freeze/thaw capabilities to enhance the recovery of products from cells and related methods.

### BACKGROUND

Bioreactors are frequently used for culturing cells. In many cases, an issue that arises is the inability to recover substantially all of the desired final product produced by the cells. This is because a substantial portion of the product (e.g., 50%) oftentimes remains trapped inside the cells, and cannot be released or retrieved, at least without opening the bioreactor or otherwise causing it to lose integrity (which could render the product worthless).

Accordingly, a need is identified for an improved bioreactor and methods for processing cells that overcome one or more of the foregoing issues, and perhaps others that have yet to be discovered. The bioreactor and methods would provide for the enhanced recovery of products of interest, that would normally remain trapped within the cells when the bioprocessing operation is completed.

### SUMMARY

According to one aspect of the disclosure, a bioreactor comprises a first chamber having cells and a second chamber providing temperature control of the cells in the first chamber. In some embodiments, the second chamber comprises a freezer connected to the bioreactor.

In some embodiments, the second chamber comprises a jacket surrounding the first chamber. The jacket may comprise an inlet and an outlet for receiving a chilled fluid, which may be sufficiently cold to cause any or all of the cells in the bioreactor to freeze. In some embodiments, the jacket comprises a pipe for circulating a chilled fluid within the jacket. The jacket may further comprise a thermally conductive fluid. The jacket may comprise a bladder including a chilled fluid for contacting a wall of the first chamber, which bladder may optionally have an inlet and an outlet. The jacket may comprise a removable sleeve. In some embodiments, the freezer comprises a chiller for chilling a fluid and a pump for circulating the fluid.

The bioreactor may further comprise a heater for heating the bioreactor. In some embodiments, the heater is adapted for at least partially receiving the bioreactor. In some embodiments, the heater comprises a conductive block having a heating element applied thereto.

In some embodiments, the heater is integrated into the bioreactor, such as by being located at least partially within a wall of the bioreactor. The heater may comprise a wire within the wall and connected to a power supply, and/or the heater may comprise a passage within the wall and connected to a source of heated fluid.

In some embodiments, the bioreactor comprises a modular bioreactor. In some embodiments, the bioreactor comprises a structured fixed bed. In some embodiments, the structured fixed bed comprises a spiral bed.

A further aspect of the disclosure pertains to a method of bioprocessing using a bioreactor including a chamber having a liquid including cells. The method comprises freezing the cells in the bioreactor.

In some embodiments, the freezing step comprises placing a chilled fluid in thermal communication with the chamber. The chilled fluid may comprise a gas. The placing step may comprise delivering the chilled fluid to a jacket of the bioreactor. The placing step may comprise delivering the chilled fluid to a pipe external to the chamber. The method may comprise the step of at least partially immersing the pipe in a thermally conductive liquid.

In some embodiments, the placing step comprises delivering the chilled fluid to a bag external to the chamber. The placing step may comprise delivering the chilled fluid to a pipe in contact with a wall of the bioreactor adjacent to the chamber. The placing step may comprise delivering the chilled fluid to a bag in contact with a wall of the bioreactor adjacent to the chamber. In some embodiments, the freezing step comprises delivering a cold gas to the chamber.

The method in some embodiments may further include the step of heating the cells after the freezing step. The heating step may comprise placing the bioreactor at least partially within a heater. The heating step may in some embodiments comprise heating a wall of the chamber. The heating of the wall may comprise supplying power to a wire on or within the wall, and/or supplying a heated fluid to a passage within the wall. The method may further optionally include monitoring the temperature of the chamber during the heating step, and/or monitoring the temperature of the chamber during the freezing step.

In some embodiments, the method further comprises generating a product within the cells prior to the freezing step. The method may then further comprise thawing the cells and recovering the product. The freezing step may comprise applying a pre-chilled sleeve to the bioreactor, such as adjacent to or around an external wall of the chamber.

According to a further aspect of the disclosure, a method of bioprocessing using a bioreactor including a chamber including cells is disclosed. The method comprises generating a product within the cells, freezing the cells, thawing the cells, and recovering the product. The disclosure is also directed to a product obtained using the aforesaid method.

This disclosure also pertains to an apparatus comprising a bioreactor including cells and means for freezing the cells. The apparatus may further include means for thawing the cells.

Still a further aspect of the disclosure pertains to an apparatus, comprising a bioreactor including a chamber having cells and a freezer connected to the bioreactor for freezing the cells in the chamber.

In some embodiments, the freezer comprises a jacket at least partially surrounding the chamber of the bioreactor. In some embodiments, the jacket comprises an inlet and an outlet for receiving a chilled fluid. The jacket may comprise a pipe for circulating a chilled fluid within the jacket. The jacket may include a thermally conductive fluid.

The jacket may comprise a bladder including a chilled fluid for contacting a wall of the chamber. The bladder may comprise an inlet and an outlet. The jacket may comprise a removable sleeve.

In some embodiments, the jacket comprises a removable sleeve. In some embodiments, the freezer comprises a chiller for chilling a fluid and a pump for circulating the fluid. The freezer may be adapted for delivering a gas to the chamber to freeze the cells. A sterile filter may be provided for sterilizing the gas prior to delivery to the chamber. A dehydrator may also be provided for drying the gas prior to delivery to the chamber.

In some embodiments, the apparatus further includes a heater for heating the bioreactor. The heater may be adapted for at least partially receiving the bioreactor. The heater may comprise a conductive block having a heating element applied thereto, or may be integrated into the bioreactor (such as by being located at least partially within a wall of the bioreactor, and may be in the form of a wire within the wall and connected to a power supply or a passage within the wall and connected to a source of heated fluid).

In some embodiments, the bioreactor comprises a modular bioreactor. In some embodiments, the bioreactor comprises a structured fixed bed, which may comprise a spiral bed.

A further aspect of the disclosure pertains to an apparatus comprising a cell culture bed including one or more frozen cells. The cell culture bed may comprise a fiber matrix. The cell culture bed may comprise a structured fixed bed.

In accordance with a further aspect of the disclosure, a modular bioreactor comprises a base, a first wall adapted for connecting with the base to form a first chamber for receiving a first fluid for culturing cells, and a second wall adapted for connecting with the base to form a second chamber for receiving a second fluid for regulating a temperature of the first chamber.

In some embodiments, the first wall comprises an inner wall and the second wall comprises an outer wall. A height of the second wall may correspond to a height of the first chamber. A freezer may also be provided for freezing the cells in the first chamber. The apparatus may further include a cooler for cooling the second fluid to an amount sufficient to cause one or more cells in the first chamber to freeze. A bladder may be provided with the second fluid for positioning in the second chamber. The first wall may include an inlet for receiving the second fluid and outlet for releasing the second fluid. The second chamber may include a coil for receiving the second fluid.

A further aspect of the disclosure pertains to a bioreactor comprising a first chamber for culturing cells, and a second chamber providing temperature control of the cells in the first chamber, the second chamber including a liquid including an anti-freezing agent.

### BRIEF DESCRIPTION OF THE DRAWING FIGURES

The novel features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 is a perspective view of a first embodiment of a bioreactor according to the disclosure.

FIGS. 2A, 2B, and 2C illustrate a possible environment of use of the bioreactor of FIG. 1.

FIG. 3 is a perspective view of the bioreactor of FIG. 1, including several enlarged views.

FIGS. 3A, 3B and 3C illustrate a matrix material for use in forming a structured fixed bed for culturing cells in any of the disclosed bioreactors.

FIG. 4 is a cross-sectional view of a second embodiment of a bioreactor according to the disclosure.

FIG. 5 is a partially cutaway view of a portion of an alternate version of the bioreactor of FIG. 4.

FIGS. 6A and 6B are cross-sectionals view a further embodiment of a bioreactor;

FIGS. 7, 8, 9, and 10 are cross-sectional views of different manners of providing a bioreactor with freeze/thaw capabilities;

FIG. 11 is a schematic diagram of another embodiment of a bioreactor with freeze/thaw capabilities; and

FIGS. 12, 13, 14, 15, and 16 illustrate various manners of facilitating the heating or thawing of a bioreactor, and the frozen cells therein in particular.

### DETAILED DESCRIPTION

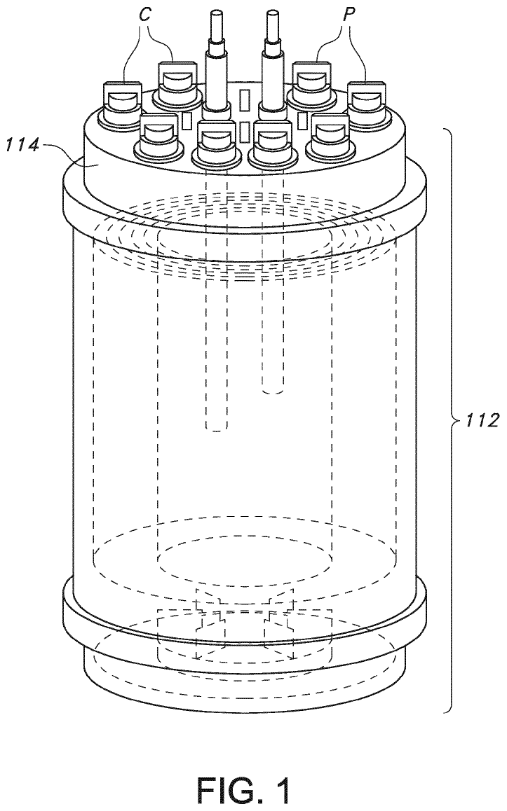

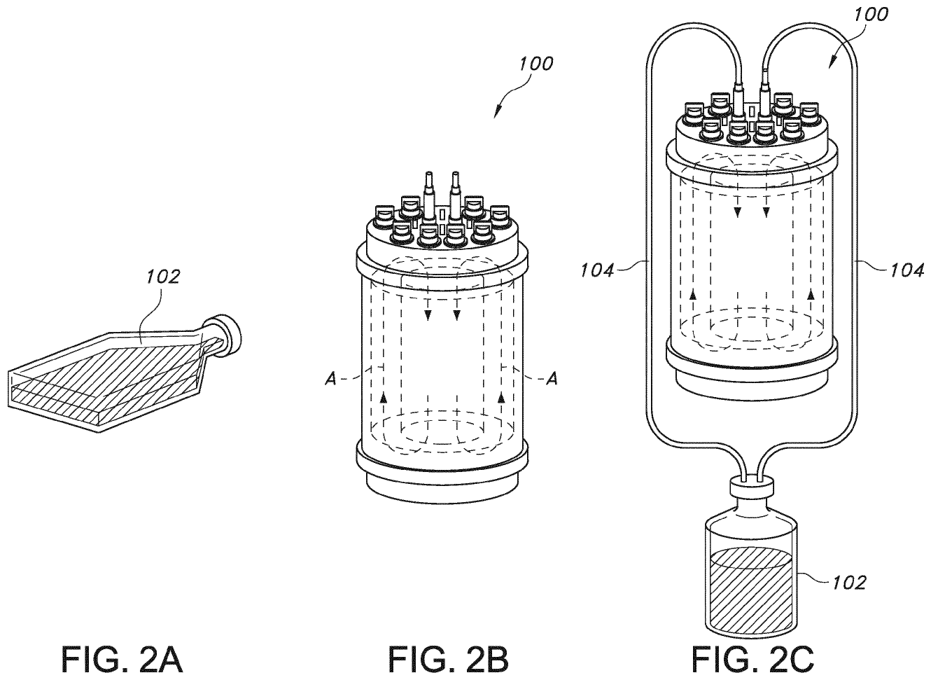

Reference is now made to FIGS. 1-3, which illustrate one embodiment of a bioreactor 100 for culturing cells, according to one aspect of the disclosure. In some embodiments, the bioreactor 100 includes an external casing or housing 112 forming an interior compartment and a removable cover 114 for covering the interior compartment, which may include various openings or ports P with removable covers or caps C for allowing for the selective introduction or removal of fluid, gas (including by way of a sparger), probes, sensors, samplers, or the like. As indicated in FIGS. 2A, 2B, and 2C, in some embodiments, the bioreactor 100 may be used in connection with an external reservoir 102 and conduits 104 (e.g., forward and return) to form a loop for circulating fluid to the bioreactor 100.

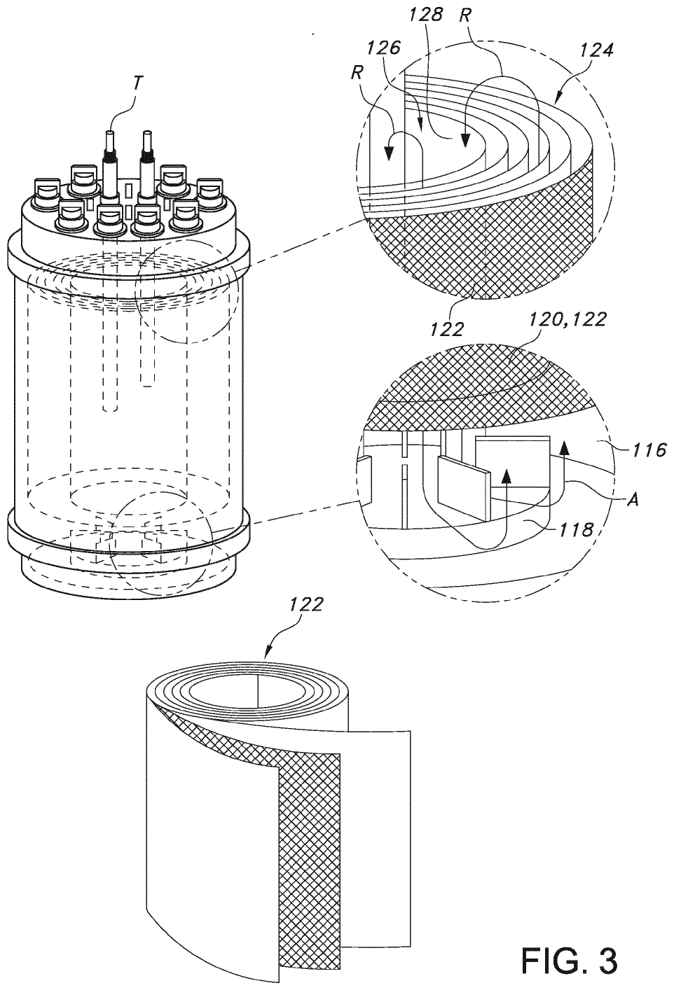

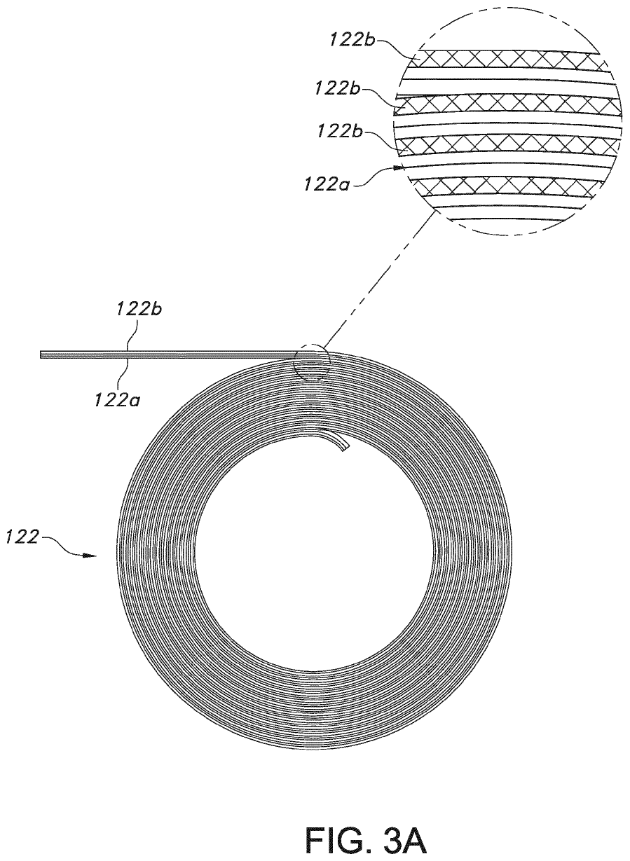

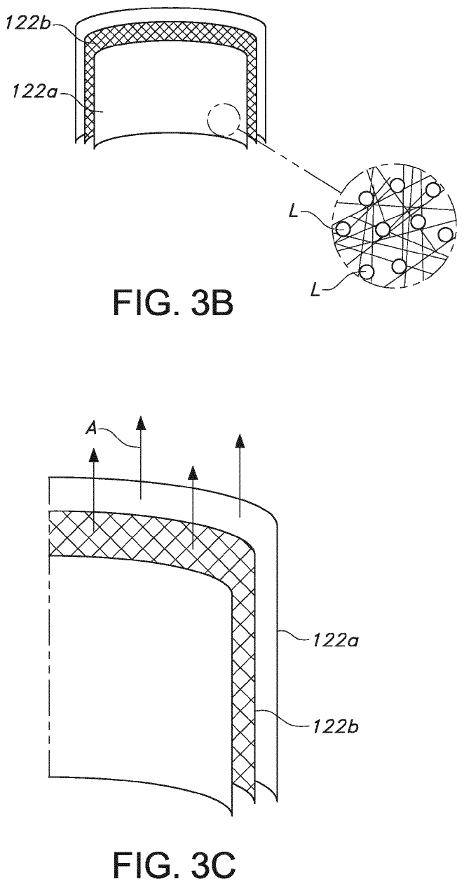

Within the interior compartment formed by the bioreactor housing 112, several compartments or chambers may be provided for transmitting a flow of fluid or gasses throughout the bioreactor 100. As indicated in FIG. 3, in some embodiments, the chambers may include a first chamber 116 at or near a base of the bioreactor 100. In some embodiments, the first chamber 116 may include an agitator for causing fluid flow within the bioreactor 100. In some embodiments, the agitator may be in the form of a “drop-in” rotatable, non-contact magnetic impeller 118 (which as outlined further below may be captured or contained within a container (not shown) including a plurality of openings for admitting and releasing fluid). In some embodiments, as a result of the agitation provided, fluid may then flow upwardly (as indicated by arrows A in FIG. 2) into an annular chamber 120 along the outer or peripheral portion of the bioreactor 100. In some embodiments, the bioreactor is adapted to receive a fixed bed, such as a structured spiral bed 122, which in use may contain and retain cells being grown. However, the bioreactor of the present disclosure can be used with any type of cell culturing arrangement, including fixed beds, packed beds, fluidized beds, or the like. As indicated in FIG. 3, in some embodiments, the spiral bed 122 may be in the form of a cartridge that may be dropped or placed into the chamber 120 at the point of use. In some embodiments, the spiral bed 122 can be pre-installed in the chamber during manufacture at a facility prior to shipping.

In some embodiments, fluid exiting the chamber 120 is passed to a chamber 124 on one (upper) side of the bed 122, where the fluid is exposed to a gas (such as oxygen or nitrogen). In some embodiments, fluid may then flow radially inwardly to a central return chamber 126. In some embodiments, the central return chamber can be columnar in nature and may be formed by an imperforate conduit or tube 128 or rather formed by the central opening of the structured spiral bed. In some embodiments, the chamber 126 returns the fluid to the first chamber 116 (return arrow R) for recirculation through the bioreactor 100, such that a continuous loop results (“bottom to top” in this version). In some embodiments, a sensor, for example a temperature probe or sensor T may also be provided for sensing the temperature of the fluid in the chamber 126. In some embodiments, additional sensors (such as, for example, pH, oxygen, dissolved oxygen, temperature) may also be provided at a location before the fluid enters (or re-enters) the chamber 116.

FIG. 3A shows one embodiment of a matrix material for use as a structured fixed bed in the bioreactor of the present disclosure and, in particular, a spiral (or “wound”) bed 122. In some embodiments, one or more cell immobilization layers 122a are provided adjacent to one or more spacer layers 122b made from a mesh structure. In some embodiments, the layering may optionally be repeated several times to achieve a stacked or layered configuration. In some embodiments, the mesh structure included in spacer layers 122b forms a tortuous path for cells for causing fluid flow radially outwardly into the immobilization layer(s) 122a (see cells L in FIG. 3B suspended or entrapped in the material of the immobilization layer 122a), and a cell culture may form part of any invention claimed herein). Homogeneity of the cells is maintained within the structured fixed bed as a result of this type of arrangement. In some embodiments, other spacer structures can be used which form such tortuous paths. In some embodiments, as shown in FIG. 3A, the structured fixed bed can be subsequently spirally or concentrically rolled along an axis or core (e.g., conduit 128, which may be provided in multiple component parts). In some embodiments, the layers of the structured fixed bed are firmly wound. In some embodiments, the diameter of the core, the length and/or amount of the layers will ultimately define the size of the assembly or matrix. In some embodiments, thickness of each of the layers 122a, 122b may be between 0.1 and 5 mm, 01 and 10 mm, or 0.001 and 15 mm.

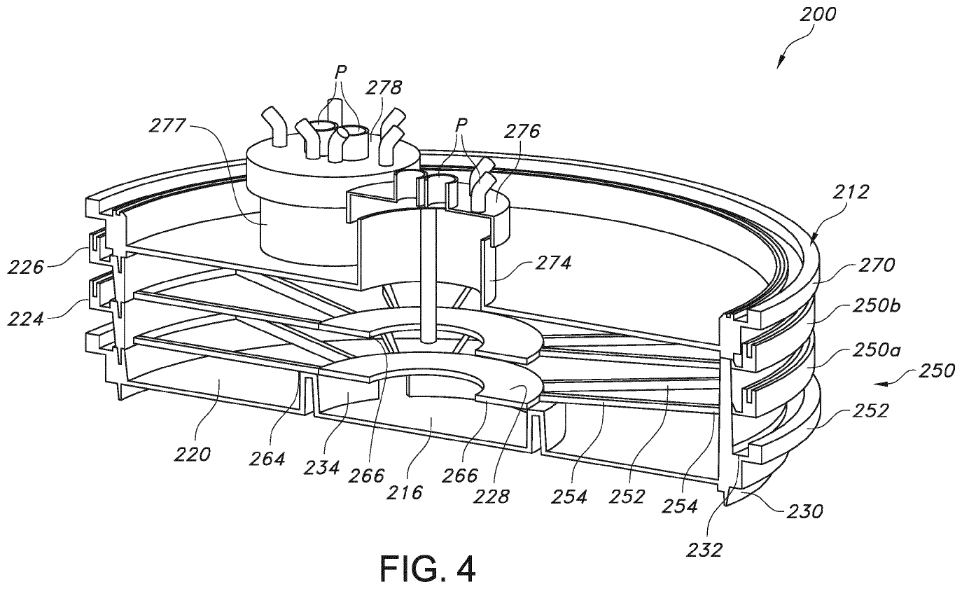

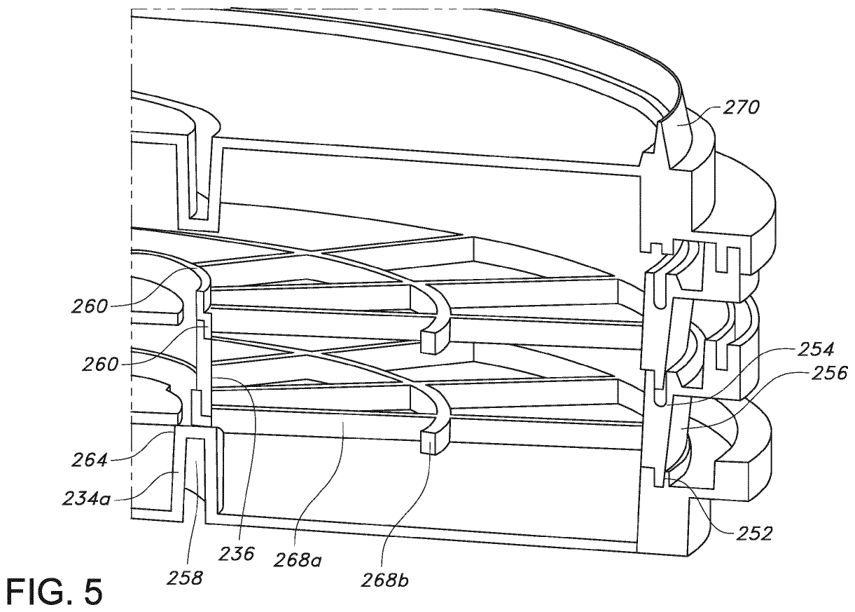

A second embodiment of a bioreactor 200 is described with reference to FIGS. 4, 5, 6A, and 6B. In some embodiments, the bioreactor 200 includes first through fifth chambers 216, 220, 224, 226, and 228 for circulating fluid in the manner described above. In this embodiment, the housing 212 is optionally comprised of a plurality of modular parts. In some embodiments, the parts include a base part 230, one or more intermediate parts 250, and a cover part 270. In some embodiments, the parts 230, 250, 270 can be adapted to interact in a fluid-tight manner so as to form the bioreactor 200 with the chambers 216, 220, 224, 226, and 228, as noted.

In some embodiments, and as perhaps best understood from FIG. 4, the base part 230 can include a peripheral connector, shown in the form of a groove 232, for receiving and engaging a corresponding peripheral connector, such as a tongue 252, projecting from one of the intermediate parts 250. In some embodiments, interiorly, the base part 230 can include an upstanding wall 234, which defines the first chamber 216 for receiving a fluid agitator (not shown). In some embodiments, the wall 234 can includes openings or passages to allow for fluid to flow radially into an outer portion of the base part 230, which defines a further or second chamber 220. In some embodiments, as the flow is redirected vertically as a result of the presence of the base part 230, turbulence is created, which thus promotes mixing and homogeneity of the fluid throughout the bioreactor and thus enhances the cell culturing process.

Two intermediate parts 250a, 250b are shown as being stacked, with a peripheral connector (groove 254) of the first (lower) part 250a engaging a corresponding connector (tongue 252) of the second (upper) part 250b. As can be appreciated from FIG. 4, in some embodiments, each intermediate part 250a, 250b can include an outer side wall 256 supporting the tongue 252 and groove 254, respectively. In some embodiments, radially inwardly, an inner wall 258 carries inner and outer connectors, which may be in the form of upstanding ledges 260, 262, can be provided for receiving the corresponding ends of a tube 236, which thus forms periphery of the fifth or return chamber 228.

In some embodiments, the first or lower intermediate part 250a may also include openings, such as elongated arcuate slots 264, which at least partially receive connectors, of the base part 230, such as upstanding projections 234a from the wall 234. In some embodiments, an interior ledge 466 can form central openings 266a in the intermediate parts 250a, 250b for permitting fluid to flow in an inner column defined by the wall 234, as well as to receive any temperature sensor, dip tube or the like (which would be positioned after the fluid exits the fixed bed). In some embodiments, the second intermediate part 250b may be similarly constructed to promote interchangeability, in which case the openings (slots 264) in the second or upper intermediate part 250b allow for the creation of the thin falling flow or film of fluid within the fifth or return chamber 228, as previously noted.

In some embodiments, extending between the inner and outer walls 256, 258 are a plurality of supports 268. In some embodiments, the supports 268 include radially extending supports 268a and at least one circumferentially extending support 268b, which together can create a perforated or reticulated plate-like structure that allows fluid flow (which structure in this or any embodiment may comprise a screen, net, grid, or other skeletal structure, and may be rigid, semi-rigid, or flexible). In fact, the supports 268 may be designed to enhance fluid flow through the bed(s) by maximizing the amount of open space created by the openings for permitting fluid to pass. In some embodiments, for culturing cells, a fixed bed, such as the spiral bed (not shown) wound around wall 234 may be positioned in the chamber 224 formed between the parts 250a, 250b. In some embodiments, fluid passing from the upper intermediate part 250b can enters the fourth chamber 226 defined partially by cover part 270 and may flow to the column forming the fifth chamber 228 before returning to the first chamber 216 for recirculation.

In some embodiments, the cover part 270 includes a connector, such as tongue 272, for fitting into the corresponding connector (groove 254) of the second intermediate part 250b. In some embodiments, the cover part 270 can also include a first or central receiver, such as upstanding wall 274 for receiving a removable cap or lid 276, which may include various ports P for connecting with conduits for delivering fluids or other substances to the bioreactor 400 (and the fifth chamber 228). In some embodiments, the cap or lid 276 may also carry the temperature sensor or probe T, as shown, as well as other sensors, and may also be adapted for providing additions or removing substances from the bioreactor 200, or for regulating a product manufacturing process. As can be appreciated, in some embodiments, the cap or lid 276 can be well positioned to allow for sensing or fluid sampling to occur in connection with the return flow via chamber 228. In some embodiments, a second peripherally positioned receiver, such as upstanding wall 277, may also be adapted for connecting with a second cap or lid 278 for receiving sensors or depositing or withdrawing substances (including culture samples) from the bioreactor and, in particular, a peripheral portion thereof including the third chamber 226 in which cell culturing is completed. In some embodiments, the caps or lids 276, 278 may have different types of ports P and may be different sizes/shapes, or they may be identical to promote interchangeability.

In some embodiments, adhesives or glue may be used at the connections to hold the structures together. In some embodiments, threaded or locking (e.g., bayonet style) connections may also be used, such that a fluid-tight seal is maintained to prevent leakage and help ensure that sterility is maintained. In some embodiments, the arrangement of modular parts 230, 250, 270 allows for the bioreactor 200 to be pre-assembled, assembled or constructed on site rapidly, and potentially disassembled with similar rapidity. As it is possible to easily add additional tube(s) to form a heightened wall 234 or intermediate parts 250, the number of fixed beds or height of the bioreactor 200 may be adjusted to suit a particular need or process setting depending on the application.

In some embodiments, the flow from one fixed bed to the next-adjacent one in the chamber is direct or uninterrupted. In some embodiments, the outer chamber 224 for receiving the bed creates a continuous flow path through the multiple beds present therein, which may be structured fixed beds, unstructured fixed beds, or other beds. In some embodiments, the continuous and substantially unimpeded flow helps to promote homogeneity as if the modules are actually a single bed and thus improves the predictability and quality of the cell culturing process. Homogeneity means that the cell distribution throughout the bed is homogeneous or having a somewhat equal spread.

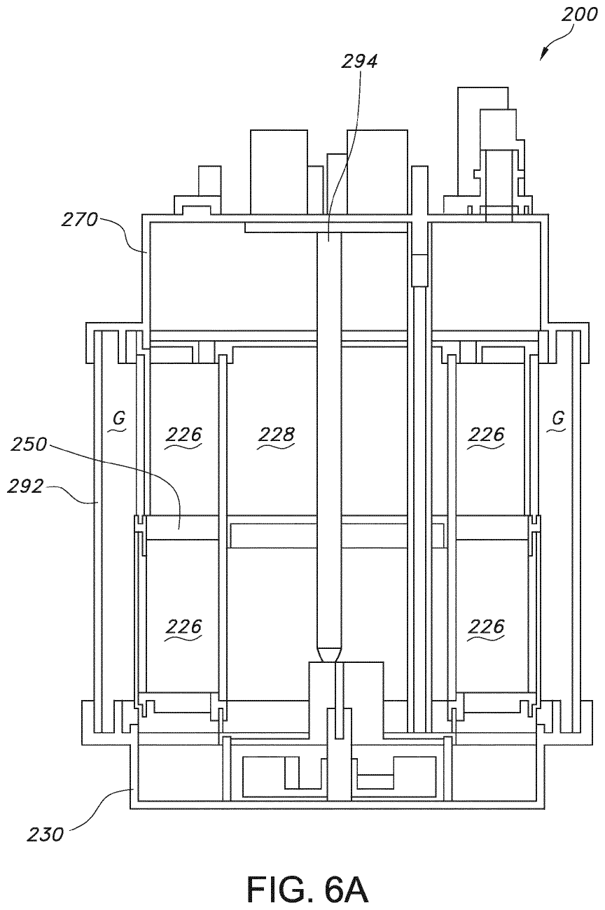

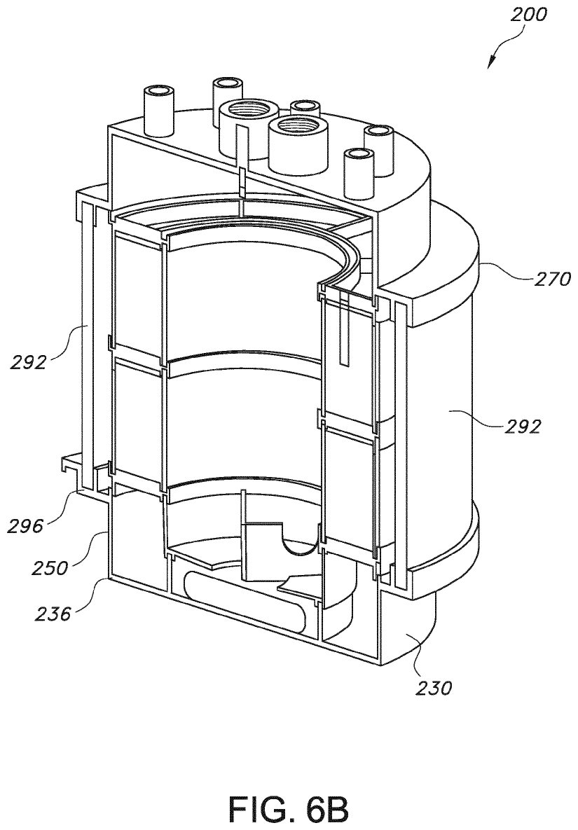

FIGS. 6A and 6B also illustrate an alternate embodiment of a modular bioreactor 200 including fixed beds 296. In some embodiments, the base part 230 and cover part 270 can be adapted for connecting with an outer casing 292, which creates a gap or space with the periphery of the intermediate parts 250. As can be understood, the height of the outer casing 292 may extend substantially the full height of the third chamber 226 for culturing cells, or it may be subdivided into individual chambers associated with each corresponding portion of the cell culture chamber. In some embodiments, the gap G or space may be used for providing a heating or cooling effect to control the temperature of the beds associated with the intermediate parts 250. The gap G or space may also simply supply insulation of the walls of the intermediate area of the bioreactor which are close to growing cells within the bed and likely to be sensitive to temperature variations. This insulation acts to prevent heat which is applied to the bottom of the base part 230 of the bioreactor from extending up to the adhered cells in the bed(s) 296.

FIG. 6A also illustrates the possible use of sparging in the bioreactor, which may be provided in any disclosed embodiment. In the illustrated arrangement, the sparging is provided by a sparger 294 located in the fifth chamber 228. The bubbles generated as a result may thus flow upwardly countercurrent to the return fluid flow.

These figures also show that the intermediate parts 250 may engage internal tubes 236, which are fluid impervious to thus provide the chamber 228 for returning flow to the base part 230, where it may be agitated and returned to enter the beds from below and flow upwardly therethrough (in any embodiment disclosed). These tubes 236 may be provided such that one tube corresponds to each fixed bed 296 present, as shown, and two intermediate parts 250 engage each tube 236 (e.g., one from below and one from above). However, in this or any other disclosed embodiment, it should be appreciated that the innermost surface of the fixed bed, such as the innermost spiral wrap of a spiral bed, may be made to perform a similar function by making it or otherwise conditioning it so as to be impervious to fluid. For instance, the surface may be coated with a fluid-impervious or hydrophobic material, such that it still retains the fluid in the bed(s) and maintains a distinct, return flow of fluid through the central column formed by chamber 228.

According to a further aspect of the disclosure, a bioreactor (which could be any of the above-described bioreactors 100, 200, or any other known form) for processing the cells to generate a product of interest (e.g., a virus or protein) may be adapted to freeze the cells in the bed to a temperature below the freezing point of the liquid in the cells (e.g. from zero degrees down to −20° C., and more preferably within the range of about −5 to about −20 degrees Celsius) and then revert to an unfrozen state (e.g., to room temperature) afterwards, by thawing (including possibly with assisted heating). This temperature cycle causes the breaking of the cell membranes and the resulting release of the final product within the cells, thereby enhancing recovery of the product of interest. This process of recovery avoids any compromising of the integrity of the bioreactor. The bioreactor may be substantially drained of liquid prior to being subjected to freezing according to the disclosure in order to avoid damage to the bioreactor that might otherwise occur upon expansion of the frozen liquid, if completely filled, but could also be subjected to freezing during the draining of the liquid).

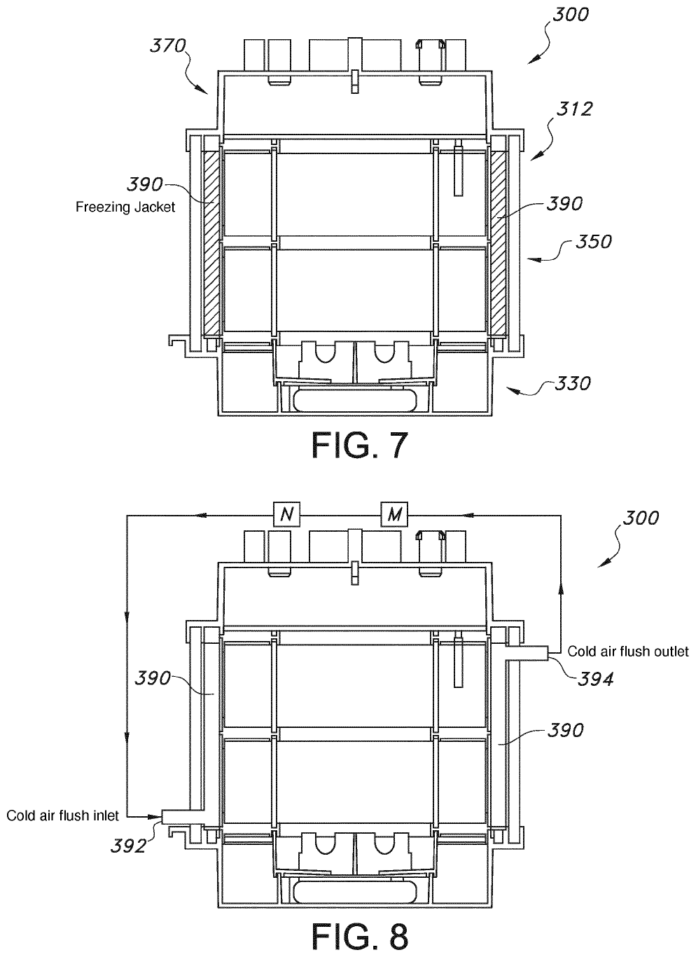

An embodiment of a bioreactor 300 is adapted to perform the above-described “freeze-thaw” operation without being placed inside of a freezer (which would be difficult, if not impossible, for most bioreactors in view of size constraints. Specifically, the bioreactor 300 may be connected directly to a freezer, the implementation of which may take various forms, as shown in FIGS. 7, 8, 9, 10, and 11, as examples. With reference to FIG. 7, the bioreactor 300 includes a housing 312 comprised of a plurality of modular parts, such as a base part 330, one or more intermediate parts 350, and a cover part 370. A jacket 390 is also provided for surrounding at least the intermediate part 350, which includes the one or more chambers for growing cells. The jacket 390 may comprise a cylindrical or annular part defining a space, which together with a chilled fluid forms an associated freezer, which may be used to thermally regulate the chamber(s) within the bioreactor 300 and associated with the intermediate part 350.

The jacket 390 may alternatively be in the form of a portable device or removable sleeve. This device or sleeve may slip over or snap around the bioreactor 300, and thus may be used when freezing is desired and not used otherwise. The sleeve can be pre-chilled/frozen, and may be reusable or made to be disposable

Causing the freezing of the liquid within the cells of the bioreactor 300 may be achieved in a variety of ways. One example is by providing a fluid (such as cold air) to the space covered by the jacket 390 (note inlet 392 and outlet 394 in FIG. 8), such that it is in direct contact with an outer surface of the bioreactor 300. The fluid may be provided by a pump M in communication with the inlet and outlet for cycling the gas through the jacket 390 in a continuous fashion, and a cooler or chiller N may serve to cool the fluid during the cycling external to the jacket 390. The jacket 390, pump M, and cooler or chiller N thus together serve as the freezer in this example.

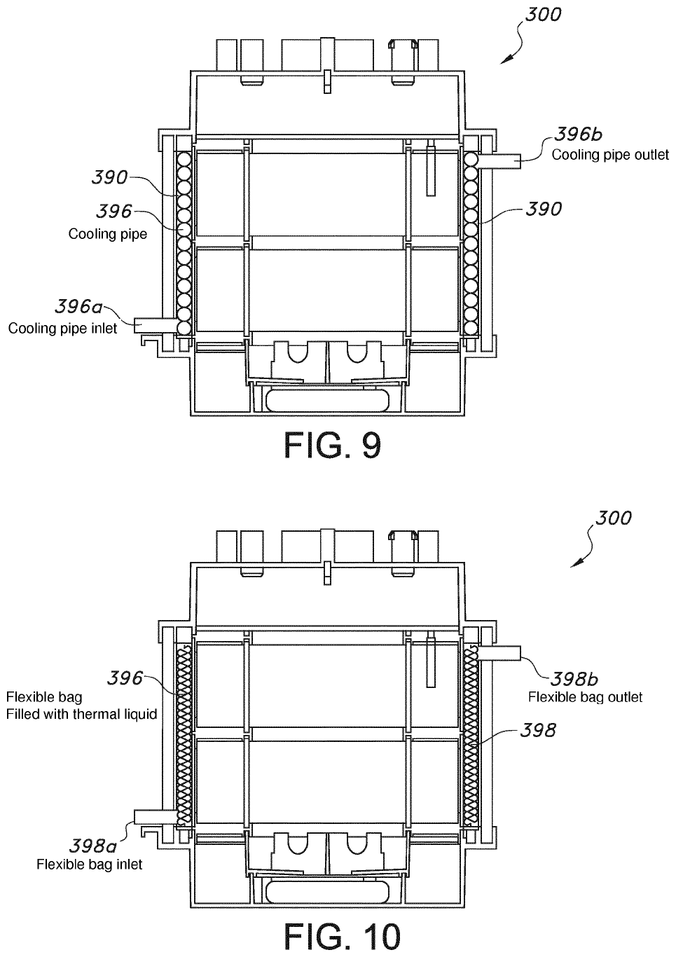

Another option, as shown in FIG. 9, is to provide a vessel for receiving a chilled fluid (e.g., an anti-freezing agent, such as liquid glycol, or water mixed with liquid glycol, and possibly with an anti-bacterial agent) in the jacket 390. This may be done by providing one or more cooling conduits, such as flexible or rigid pipes 396, in the space bounded by the jacket 390. Each pipe 396 provided may also have an inlet and outlet 396a, 396b for circulating a fluid in a circuit with a freezer and pump (see FIG. 8). Using more than one pipe (such as in a stacked configuration) is possible and may allow for a better degree of thermal control and avoid possible “hot” spots by staggering the location of the respective inlet and outlet. The pipe(s) 396 may also be immersed in a fluid within jacket 390 serving as a good thermal conductor (e.g, liquid glycol) to improve thermal conductivity and allow for an enhanced degree of thermal transfer. In the typical case where the bioreactor 300 is cylindrical, the pipes 396 would be annular in nature, and may traverse the bioreactor 300 in a helical fashion. As discussed below, the pipes 396 may also be integrated into one or more walls of the bioreactor 300.

Instead of a pipe(s) 396, the vessel may comprise a bladder, such as one or more flexible bags 398, as shown in FIG. 10. The bag 398 may also have an inlet 398a and outlet 398b for circulating chilled fluid. In view of the large surface area and flexible surface of the bag 398, a better degree of thermal transfer may result due to the increase area of surface contact with the outer surface of the intermediate part 350. Again, for the typical cylindrical bioreactor 300, the bag 398 provides a generally annular shape so that the respective surfaces are in intimate thermal engagement to maximize the transfer of cooling. Alternatively, the bladder or bag 398 may not have an inlet or outlet and may have its temperature regulated by integrating a heating or cooling system into adjacent structure, such as for example the intermediate part 350 or another part. In any case, the bladder or bag may comprise a multilayer flexible bag made of polymer material (HDPE or PVC, as examples) designed to withstand freezing or cyclic temperature changes, and can be designed to fit around or within a chamber of any bioreactor according to the needs of the particular arrangement.

In any of these cases or others where the liquid in or around the cells is frozen, it may be allowed to thaw naturally by simply stopping the provision of chilled fluid to the jacket 390. Alternatively, a warming fluid may be supplied to the jacket 390 to assist with the thawing procedure. Still another alternative, and as discussed further below, is to use a heater to assist in the thawing operation for the frozen (or partially frozen) cells, which may involve adding a warm fluid or buffer to the jacket 390 or the bioreactor 300 itself.

Upon thawing, the cell membranes of the once-frozen cells are broken. Fluid such as buffer may be introduced to the bioreactor 300 to harvest the product or material of interest (e.g., a virus or protein). The bioreactor 300 may be at least partially drained of fluid before the freezing step, thereby creating room for additional fluid (i.e., the buffer, which again may be warmed to facilitate thawing). Once liquefied, the fluid may be pumped out of the bioreactor 300 to a holding tank and may be purified thereafter (either separate of combined with the thawed fluid).

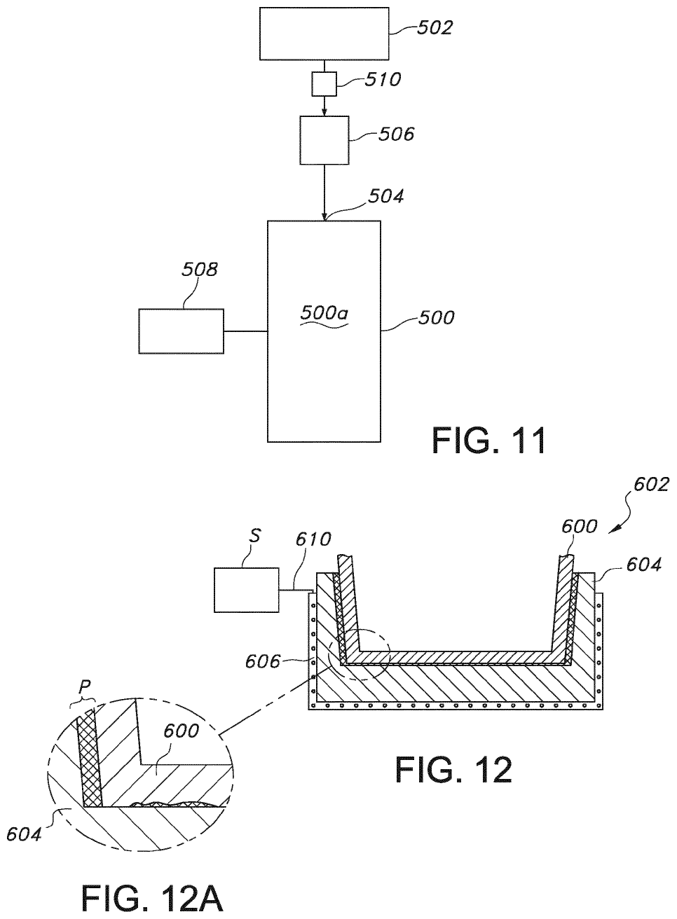

According to another aspect of the disclosure, and referring to FIG. 11, cells can be frozen by providing a cold medium to a bioreactor 500, such as cold air at, for example, from −5 degrees C. to about −20 degrees C. or otherwise at a temperature sufficient to cause any or all cells to freeze. As indicated schematically in FIG. 11, this can be achieved by using a freezer 502 to supply the cold medium directly to the bioreactor 500, and a chamber 500a thereof in which cells are cultured in particular, via an inlet 504 or other port, and may optionally be done with a sterilization filter 506, which may be upstream or downstream of the freezer 502. This is contrasted with the above embodiment in which an outer chamber of the bioreactor is used to receive a fluid for causing freezing of the cells. A temperature sensor or probe 508 may also be provided to monitor the temperature of the bioreactor 500 and, in particular, any portion including cells to be frozen.

If air is used as the medium for causing the cells to freeze, it may optionally be dehydrated so as to avoid having moisture condense and freeze in any delivery conduit and the sterilization filter 506, if present, and render both components unusable. Dehydrated air can be provided by drying ambient air using a dehydrator 510, as indicated, which make take the form of molecular sieves, a heat exchanger or other heat source, and which may be upstream or downstream of the freezer 502. Additionally, the air may be dried prior to delivery (before or after being chilled) by exposing it to coils or some other source at a temperature that is below the air target temperature of −20 degrees C., i.e. −30 degrees C. Instead of air, other gases could also be used.

As noted above, subsequent to freezing the cells, it may be desirable to subject the cells to thawing. This may be achieved passively or naturally by simply withdrawing any cold influence from an associated freezer or freezing means and waiting. However, it is also possible to subject the cells to active thawing, such as by warming or heating the cells or an associated portion or chamber of the bioreactor.

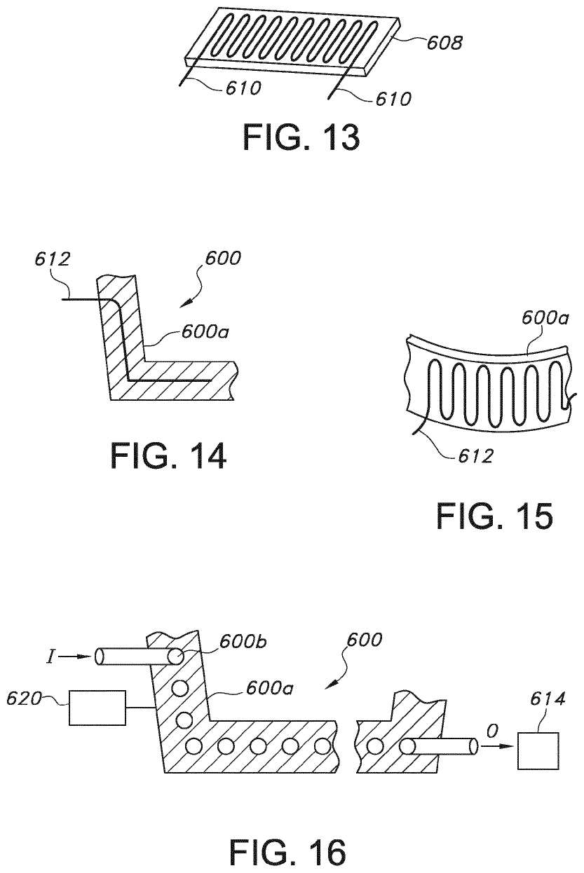

In some embodiments, active heating may be achieved using externally applied or transferred heat, and various examples of achieving such heating are shown in FIGS. 12-16. In FIG. 12, a heat source or heater 602 for a bioreactor 600 comprises a conductive block 604. This block 604 may be formed of metal and is adapted for receiving at least a portion of the bioreactor 600 (such as a base thereof). A heating element 606 may be applied to the block 604, which as shown in FIG. 13 may comprise a flexible strip, such as one made of a polymer film 608 on or in which an electric wire 610 is attached (e.g., glued or encapsulated), and which wire may be connected to a power supply S. Alternatively, the block 604 may be provided with one or more passages (not shown) for receiving a flow of fluid. In any case, the bioreactor 600 may be associated with the heater 602 to achieve the desired heat transfer, and cause the more rapid thawing of the once-frozen cells or portions of the bioreactor 600.

As can be appreciated from FIGS. 12 and 12A, a gap P may exist between the bioreactor 600 and the recessed portion of the block 604 receiving it, which can result in suboptimal heat transfer. Thus, according to a further aspect of the disclosure, and with reference to FIGS. 14, 15, and 16, the heater 602 may be instead or additionally integrated directly into the bioreactor 600 (which may take any form disclosed herein or otherwise). FIG. 14 illustrates that a resistive heating element, such as an electrically conductive wire 612, may be integrated into the wall 600a of the bioreactor 600, such as by injection molding (and overmolding in particular, so that intimate contact with the wire and the material (plastic) of the bioreactor wall 600a results). Alternatively or additionally, the wire 612 may be externally applied to the wall 600a as indicated in FIG. 15, and in a manner that maximizes the heating effect (such as by applying it in a serpentine pattern, as shown, but any pattern that maximizes the coverage area could be used). As can be appreciated, this allows for the heating to be selectively applied to portions of the bioreactor 600 in a more precise manner, depending on the cell culture process and/or where the heating may be desired to maximize efficiency (e.g., only on portions of the bioreactor corresponding to a chamber where cells are cultured).

Referring to FIG. 16, a further option for temperature regulation to achieve active thawing is to integrate a passage 600b in a wall 600a of the bioreactor 600. A fluid (gas or liquid) may be passed through the passage 600b to achieve the desired the thermal exchange (heating or cooling). In particular, the fluid may be caused to flow from an inlet I to and outlet O, and possibly in a recirculating loop in communication with a heater 614. A temperature probe or sensor 620 may also be provided to monitor the temperature of the bioreactor 600 in a more accurate manner and helps to ensure that overheating and damage does not result.

This disclosure may also be considered to relate to a cell culturing bed with frozen cells, whether part of a bioreactor or otherwise.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, preferably +/−10% or less, more preferably +/−5% or less, even more 35 preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

While preferred embodiments of the present invention have been shown and described herein, it will be obvious to those skilled in the art that such embodiments are provided by way of example only. Numerous variations, changes, and substitutions will now occur to those skilled in the art without departing from the invention. For example, while the bioreactor is shown in a vertical orientation, it could be used in any orientation. Also, while the bioreactor is shown throughout independent of any isolator or cabinet, it should be understood that it could be used in combination with such structures in order to maintain a sterile environment. It should also be understood that various alternatives to the embodiments of the invention described herein may be employed in practicing the invention. It is intended that the following claims define the scope of the protection under the applicable law and that methods and structures within the scope of these claims and their equivalents be covered thereby.

1. A bioreactor, comprising:
a first chamber having cells; and
a second chamber providing temperature control of the cells in the first chamber.

2. The bioreactor of claim 1, wherein the second chamber comprises a freezer connected to the bioreactor.

3. The bioreactor of claim 1, wherein the second chamber comprises a jacket surrounding the first chamber.

4. The bioreactor of claim 3, wherein the jacket comprises an inlet and an outlet for receiving a chilled fluid.

5. The bioreactor of claim 3, wherein the jacket comprises a pipe for circulating a chilled fluid within the jacket.

6. The bioreactor of claim 5, wherein the jacket includes a thermally conductive fluid.

7. The bioreactor of claim 3, wherein the jacket comprises a bladder including a chilled fluid for contacting a wall of the first chamber.

8. The bioreactor of claim 7, wherein the bladder comprises an inlet and an outlet.

9. The bioreactor of claim 2, wherein the jacket comprises a removable sleeve.

10. The bioreactor of claim 2, wherein the freezer comprises a chiller for chilling a fluid and a pump for circulating the fluid.

11. The bioreactor of claim 1, further comprising a heater for heating the bioreactor.

12. The bioreactor of claim 11, wherein the heater is adapted for at least partially receiving the bioreactor.

13. The bioreactor of claim 11, wherein the heater comprises a conductive block having a heating element applied thereto.

14. The bioreactor of claim 11, wherein the heater is integrated into the bioreactor.

15. The bioreactor of claim 14, wherein the heater is located at least partially within a wall of the bioreactor.

16. The bioreactor of claim 15, wherein the heater comprises a wire within the wall and connected to a power supply.

17. The bioreactor of claim 15, wherein the heater comprises a passage within the wall and connected to a source of heated fluid.

18. The bioreactor of claim 1, wherein the bioreactor comprises a modular bioreactor.

19. The bioreactor of claim 1, wherein the bioreactor comprises a structured fixed bed.

20.-43. (canceled)

44. An apparatus, comprising:
a bioreactor including a chamber having cells; and
a freezer connected to the bioreactor for freezing the cells in the chamber.

45.-77. (canceled)
